(19) United States
(12) Patent Application Publication
(10) Pub. No.: US 2005/0159728 A1
(43) Pub. Date: Jul. 21, 2005

(54) Steerable sheath

(75) Inventors: Andrew W. Armour, Media, PA (US); Brandon N. Beck, Westville, NJ (US)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: Thomas Medical Products, Inc., Malvern, PA (US)

(21) Appl. No.: 11/033,915

(22) Filed: Jan. 12, 2005

Related U.S. Application Data
(60) Provisional application No. 60/536,588, filed on Jan. 15, 2004.

(51) Int. Cl. A61M 25/01

(52) U.S. Cl. 604/528; 600/434; 606/108

(57) ABSTRACT

A steerable sheath having an inner catheter that can slide and rotate within an outer rigid composite catheter and whereby an entire range of curvatures can be formed by the distal end of the inner catheter through sliding and/or rotating the inner catheter within the outer catheter. The inner catheter can be releasably locked within the outer catheter. Curvature indicia are provided on the inner sheath to facilitate creating a particular three-dimensional curve. Apertures are provided in the inner sheath to permit flushing the device with a single flushing source.

### CROSS-REFERENCE TO RELATED APPLICATIONS

This utility application claims the benefit under 35 U.S.C. §119(e) of Provisional Application Ser. No. 60/536,588 filed on Jan. 15, 2004 entitled STEERABLE SHEATH and whose entire disclosure is incorporated by reference herein.

### BACKGROUND OF THE INVENTION

1. Field of Invention

This invention relates generally to catheters and, more particularly, to steerable catheters.

2. Description of Related Art

Examples of steerable catheters are described in U.S. Pat. No. 6,616,628 (Hayzelden); U.S. Pat. No. 6,610,058 (Flores); U.S. Pat. No. 6,607,496 (Poor et al.); U.S. Pat. No. 6,592,581 (Bowe); U.S. Pat. No. 6,146,355 (Biggs); and U.S. Pat. No. 5,636,634 (Kordis et al.), as well as in U.S. Patent Application Publication No. 2003/0109861 (Shimada). See also European Patent Application Nos. 0745407 (Daig Corporation) and 0605796 (C.R. Bard, Inc.).

Examples of catheter exchange devices, as well as shaft control and handle control mechanisms used with steerable catheters are shown in U.S. Pat. No. 6,371,940 (Valencia et al.); U.S. Pat. No. 5,693,021 (Diaz et al.); U.S. Pat. No. 5,588,442 (Scovil et al.); U.S. Pat. No. 5,449,362 (Chaisson et al.); U.S. Pat. No. 5,388,590 (Horrigan); and U.S. Pat. No. 5,318,527 (Hyde et al.), as well as in U.S. Patent Application Publication Nos. 2002/0165484 (Bowe et al.) and 2001/0027323 (Sullivan, III et al.). See also European Patent Application No. 1019133 (C.R. Bard, Inc.).

However, there remains a need to provide a steerable catheter that can be manipulated by the surgeon to form a desired curvature without the need to use preformed introducers and to also provide a steerable catheter whereby the inner sheath can be releasably locked within the outer sheath. There also remains a need to provide the surgeon with indicia on the steerable catheter for forming a particular curvature. There also remains a need for permitting a single pressure source for flushing the steerable catheter.

All references cited herein are incorporated herein by reference in their entireties.

### BRIEF SUMMARY OF THE INVENTION

A steerable sheath for introducing an elongated instrument therethrough into the body of a living being comprising: a first catheter and a second catheter, wherein the first catheter is an elongated flexible member formed of a first material (e.g., poly ether block amide such as PEBAX® or other polymer) and has a first distal end and a lumen extending therethrough, a portion of the first catheter adjacent the first distal end is of a first radius of curvature; the second catheter is an elongated flexible member formed of a composite material (e.g., Nylon 12, 30% glass bead, or Nylon 12, 30% Calcium Carbonate, or Nylon 12, 25% Talc, 12% Barium Sulfate, or Nylon 12, 25% Talc, 10% Barium Sulfate, or Nylon 6, 5% clay nanocomposite, or Nylon 12, 7% clay nanocomposite, or Nylon 12, 8% clay nanocomposite, Polyester/LCP blend, etc.) and has a longitudinal axis, a second distal end and a lumen extending therethrough; and the first catheter is located within the lumen of the second catheter, wherein the first and second catheters are arranged to be coupled together so that the first distal end projects from the second distal end and the first catheter can be rotated about and slid longitudinally with respect to the longitudinal axis, wherein the composite material is more rigid than the first material, whereupon the first distal end is able to form a plurality of curve shapes when the first catheter is slid and/or rotated within the second catheter.

A steerable sheath comprising: a first catheter including a first lumen and a second catheter including a second lumen, wherein the second catheter is slidable and rotatable within the first lumen; and a locking mechanism (e.g., a rack and pinion configuration and a compressible gland, etc.) coupled to the first catheter and to the second catheter, wherein the locking mechanism prevents or permits the first catheter to slide or rotate within the second catheter.

### BRIEF DESCRIPTION OF SEVERAL VIEWS OF THE DRAWINGS

The invention will be described in conjunction with the following drawings in which like reference numerals designate like elements and wherein:

FIG. 1 is an isometric view of the steerable sheath of the present invention coupled to a conventional hemostasis valve and stop cock during use and which shows the curvature of the inner sheath as well as the curvature of the outer sheath;

FIG. 2 is another isometric view of the steerable sheath of the present invention coupled to a conventional hemostasis valve and stop cock during use and which depicts a three-dimensional angle of the inner sheath with respect to the outer sheath;

FIG. 3 is an isometric view of the inner sheath only and which also includes cross-sectional views taken at two different locations along the length of the inner sheath;

FIG. 4 depicts a set of conventional introducers that are pre-formed into particular fixed curvatures;

FIG. 5 is an end view of the proximal end of the present invention taken along line 5-5 of FIG. 2;

FIG. 6A is a cross-sectional view of the lock mechanism of the present invention taken along line 6A-6A of FIG. 5 and showing the lock mechanism in an unlocked state, thereby permitting the inner sheath to be displaced within the outer sheath;

FIG. 6B is a cross-sectional view of the lock mechanism of the present invention taken along line 6B-6B of FIG. 5 and showing the lock mechanism in locked state, thereby preventing the inner sheath from being displaced within the outer sheath; and

FIG. 7 is a top partial view of the present invention showing lock/unlock indicia on the locking mechanism as well as alignment indicia on the inner sheath for positioning in a window portion of the lock mechanism housing.

### DETAILED DESCRIPTION OF THE INVENTION

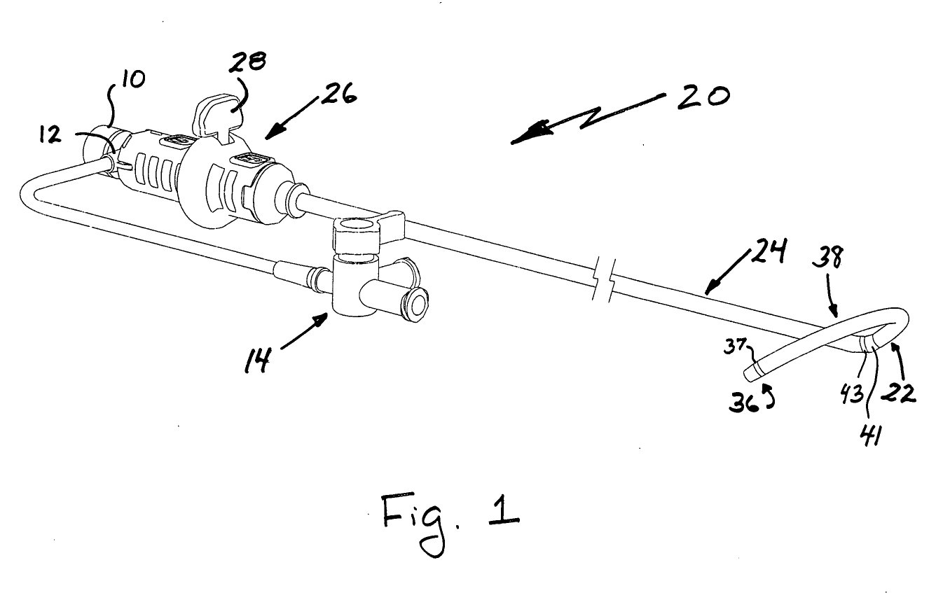

There is shown in FIG. 1 a steerable sheath system 20 in accordance with the present invention. The steerable sheath 20 operates as an access device, including supporting the introduction of vascular instruments therein, for entering a vein or an artery and for delivering another catheter therethrough to a particular target site within the cardiovascular system. Thus, the steerable sheath 20 provides for delivering a catheter within the body, e.g., into the heart, and addresses the electrophysiologist's needs for addressing atrial fibrillation or atrial flutter following transseptal placement through the fossa ovalis region (i.e., the small membrane that separates the right and left atrium). Since the present invention 20 permits an inner sheath having a first radius of curvature to be slid and/or rotated within an outer sheath of a rigid material, an entire range of curves (including three-dimensional curves) can be achieved using this invention, thereby eliminating the need for customized introducers having fixed radii of curvatures.

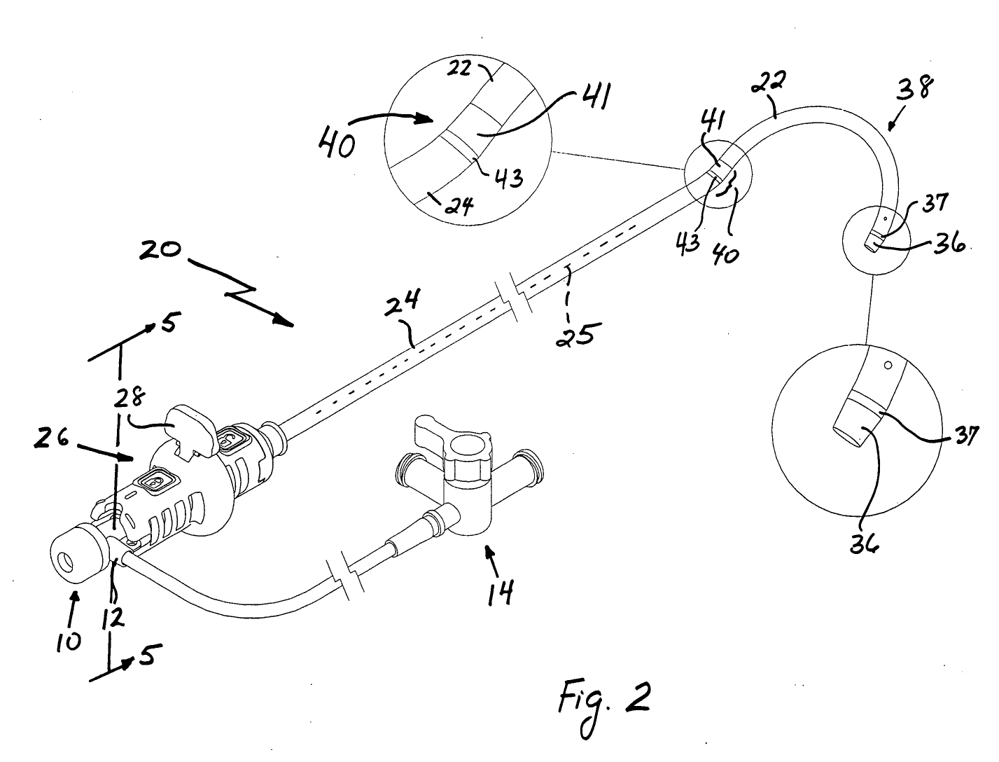

The steerable sheath 20 comprises an inner catheter or sheath 22 and an outer catheter or sheath 24 wherein the inner sheath 22 can slide and rotate within the outer sheath 24. Each catheter 22/24 comprises an elongated flexible member having a lumen therethrough. A locking mechanism 26 having a control lever 28 controls the ability to slide and/or rotate the inner sheath 22 within the outer sheath 24. During use of the invention 20, the proximal end (most clearly seen in FIG. 2) of the inner sheath 22 may be coupled to a conventional hemostasis valve 10 which in turn is connected through a side-port 12 to a stop cock 14.

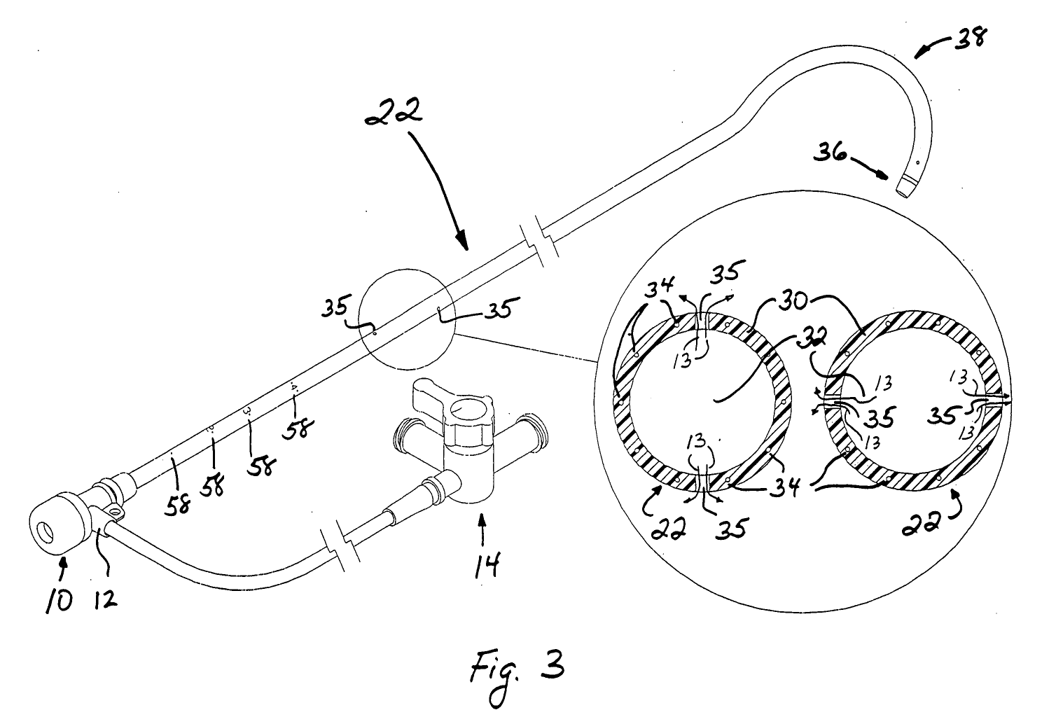

As shown in FIG. 3, the inner sheath 22 comprises a tubular construction having a layer 30 (e.g., poly ether block amide such as PEBAX® or other polymer) that forms a central passageway/lumen 32 to allow for passage of the vascular instruments (not shown). Embedded within the layer 30 are braids or strands of reinforcing stainless steel (e.g., 304 stainless steel) 34. The braided construction provides sufficient torqueability to permit the inner sheath 22 to be manipulated by the surgeon during use. As will be discussed in detail later, a portion of the inner sheath 22 comprises a plurality of apertures 35 that permit the passage of fluid therethrough in order to fill the space between the inner sheath 22 and the outer sheath 24, when the inner sheath 22 is disposed therein. It should be understood that although a plurality of apertures 35 are shown, the presence of at least one aperture 35 does suffice. The open tip 36 (FIGS. 1-3) of the inner sheath 22 comprises an atraumatic tip for safety and radiopaque section 37 for facilitating imaging during use (e.g., X-ray, fluoroscope, etc.). The distal end 38 (FIG. 1) of the inner sheath 22 comprises a first radius (e.g., 1 1/4″) of curvature (e.g., 180°), which when passed through the outer sheath 24, can assume a plurality of different curvatures, including three-dimensional curves.

The outer sheath 24 comprises a rigid material that can alter the curvature of the distal end 38 of the inner sheath 22 when that distal end 38 passes through the distal end 40 of the lumen (not shown) of the outer sheath 24. The outer sheath 24 has a longitudinal axis 25 (FIG. 2). One of the important features of the present invention 20 is the rigidity of the outer sheath 24 while minimizing any increase in the diameter of the outer sheath 24 in order to permit passage of the invention 20 through the body vessel. Thus, Applicants have determined that by using a composite material, a rigid outer sheath can be obtained while utilizing a relatively thin wall (e.g., two to eight thousandths of an inch). Examples of such composite material that exhibit these qualities and which do not kink are:
Nylon 12, 30% glass bead
Nylon 12, 30% Calcium Carbonate
Nylon 12, 25% Talc, 12% Barium Sulfate
Nylon 12, 25% Talc, 10% Barium Sulfate
Nylon 6, 5% clay nanocomposite
Nylon 12, 7% clay nanocomposite
Nylon 12, 8% clay nanocomposite
Polyester/LCP blend
   Of these polymers and additives, one particular effective material is Nylon 12, 25% Talc, 10% Barium Sulfate.

Furthermore, the distal end 40 of the outer sheath 24 may also include a radius of curvature, similar or different, from the radius of curvature of the inner sheath 22. By way of example only, FIGS. 1 and 2 depict a radius of curvature for the outer sheath 24 in the range of 30°-50°. However, it should be understood that this is by way of example only and that any radius of curvature, or no radius of curvature at all (e.g., linear), can be used with the distal end 40 of the outer sheath 24. The tip 41 (FIG. 1) of the outer sheath 24 also comprises an atraumatic tip for safety and radiopaque section 43 for facilitating imaging during use (e.g., X-ray fluoroscope, etc.).

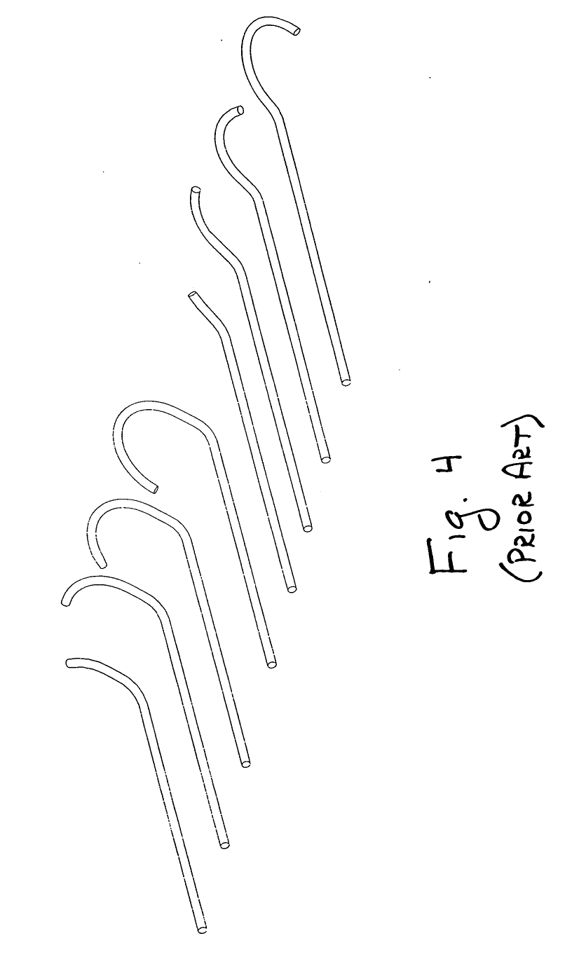

By manipulating the inner sheath 22 within the outer sheath 24, the distal end 38 of the inner sheath 22 can be formed into an entire range of curves, both two-dimensional and three-dimensional. FIG. 4 depicts conventional introducer distal sections that are pre-formed into particular curvatures. Thus, once a surgeon determines which curvature best serves the need for a particular instrument feed, one of the these introducers is selected. In contrast, the present invention 20 can be formed into any of these curvatures, as well as any curvature that falls between the ones shown in FIG. 4. As a result, the present invention 20 eliminates the need to have pre-formed introducers by allowing the surgeon to create the particular curvature he/she needs. This feature is extremely important due to the differences in anatomical location and size among different patients. Having a catheter that can be modified, on site, to form a particular curvature, increases surgical efficiency while reducing patient trauma, infection and instrument costs.

It is important to prevent air from entering the present invention 20 and making its way between the sheaths 22/24, as well as preventing blood from finding an egress through the present invention 20. In conventional practice for any body-inserted medical device, it is necessary to flush the air out that may be trapped/present in the device; in medical devices having coaxial-displaceable members, such as the present invention 20, the annular space between the inner sheath 22 and the outer sheath 24 must be flushed of air. Currently, in medical devices having an inner displaceable member, separate flush sources must be used to flush the inner and outer members separately. In contrast, as mentioned earlier, the inner sheath 22 comprises a plurality of apertures 35, thereby providing fluid communication with the lumen of the outer sheath 24. Thus, when flushing the present invention 20 using a solution (e.g., a sterile saline solution), the solution is injected into the inner sheath 22 via the hemostasis valve 10. As shown in FIG. 3, the solution 13 passes through the passageway 32 of the inner sheath 22, the solution 13 passes through the apertures 35 and into the passageway of the outer sheath 24, i.e., the annular space between the outside of the inner sheath 22 and the inside surface of the outer sheath 24. As the solution 13 fills the annular space, it displaces the air out of the present invention 20. As a result, the present invention 20 is able to be flushed using a single flush source in fluid communication with the inner sheath 22. It should be understood that although a plurality of apertures 35 is shown in the inner sheath 22, a single aperature 35 would suffice. Moreover, the size of each aperture is smaller than the diameter of conventional guide wires to prevent the end of the guide wire that may be inserted in the present invention 20 from getting lodged inside any of these apertures 35.

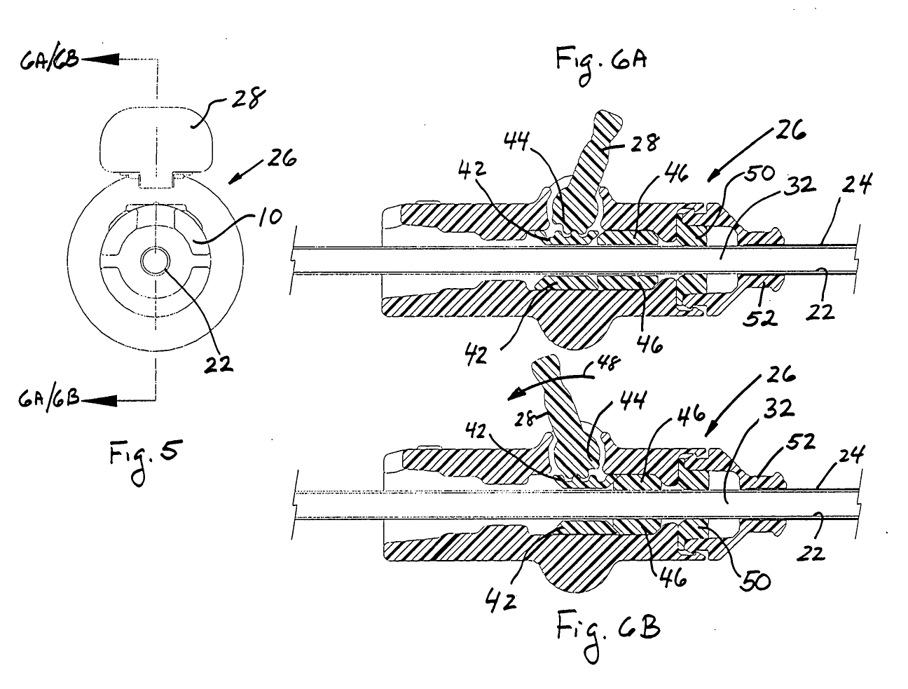

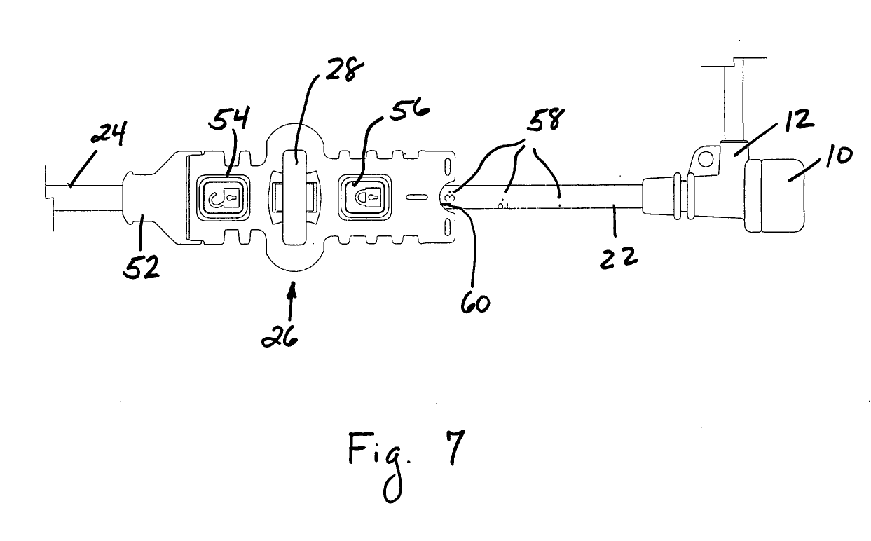

Another unique feature of the steerable sheath 20 is the ability to releasably lock the inner sheath 22 and the outer sheath 24 together, and without crimping or collapsing the inner sheath 22. By way of example only, there is shown in FIG. 5 a locking mechanism 26 that provides for the releasable lock of the inner sheath 22 with the outer sheath 24. In particular, the lock mechanism comprises a rack 42 and pinion gear 44 having the control lever 28 integrally-formed therewith, as shown most clearly in FIG. 6A. An elastomeric gland 46 forming a tight fit around the inner sheath 22 and which permits the inner sheath 22 to slide/rotate within the outer sheath 24 when the lock mechanism 26 is unlocked, as shown in FIG. 6A. When the control lever 28 is rotated counterclockwise (see direction of arrow 48 in FIG. 6B), the pinion gear 44 displaces the rack 42 to the right (FIG. 6B), compressing the gland 46 (note the size of the gland in FIG. 6A with that shown in FIG. 6B) causing it to flex inward and seize or lock the inner sheath 22, thereby preventing the inner sheath 22 from any displacement/rotation within the outer sheath 24. It should be noted that this clamping of the inner sheath 24 does not crimp or collapse the inner sheath 22, thereby maintaining the opening of the passageway 32. An elastomeric seal 50 surrounds the inner sheath 22 inside the locking mechanism 26 housing to prevent air or fluid from entering the annular space between the sheaths 22/24 via the locking mechanism 26 housing, and thereby preventing entry of an air embolism or blood from emanating outside of the outer sheath 24. One end of the outer sheath 24 is fixedly secured (e.g., bonded) to end portion 52 of the locking mechanism 26. It should be understood that this preferred lock mechanism 26 is by way of example and does not limit the scope of the invention in any way to the releasable lock mechanism shown therein and includes lock mechanisms such as those shown in corresponding A. Ser. No. 60/536,588 whose entire disclosure is incorporated by reference herein.

FIG. 7 shows the top view of the locking mechanism 26. Unlock indicium 54 and lock indicium 56 provide the operator of the present invention 20 with a quick alert as to the displacement status of the inner sheath 22. Furthermore, to assist the operator in creating a particular three-dimensional curve, curve indicia 58 are provided on a portion of the inner sheath 22. Thus, when the operator displaces longitudinally and/or rotates the inner sheath 22 and positions one of the particular curve indicia 58 in the window 60, a particular three dimensional curve is formed at the tip 38 of the inner sheath 22. Thus, this unique feature permits the operator to quickly configure the inner sheath tip 38 to a particular three dimensional curve 38.

While the invention has been described in detail and with reference to specific examples thereof, it will be apparent to one skilled in the art that various changes and modifications can be made therein without departing from the spirit and scope thereof.

1. A steerable sheath for introducing an elongated instrument therethrough into the body of a living being comprising:
a first catheter and a second catheter, said first catheter being an elongated flexible member formed of a first material and having a first distal end and a lumen extending therethrough, a portion of said first catheter adjacent said first distal end being of a first radius of curvature;
said second catheter being an elongated flexible member formed of a composite material and having a longitudinal axis, a second distal end and a lumen extending therethrough; and
said first catheter being located within said lumen of said second catheter, said first and second catheters being arranged to be coupled together so that said first distal end projects from said second distal end and said first catheter can be rotated about and slid longitudinally with respect to said longitudinal axis, said composite material being more rigid than said first material, whereupon said first distal end is able to form a plurality of curve shapes when said first catheter is slid and/or rotated within said second catheter.

2. The steerable sheath of claim 1 wherein a portion of said second catheter adjacent said second distal end is linear or has a second radius of curvature, said second radius of curvature being different from said first radius of curvature.

3. The steerable sheath of claim 1 wherein a portion of said second catheter adjacent said second distal end is linear or has a second radius of curvature, said second radius of curvature being similar to said first radius of curvature.

4. The steerable sheath of claim 1 wherein said first and second catheters are coupled to a locking mechanism, said locking mechanism preventing or permitting said first catheter to slide or rotate within said second catheter.

5. The steerable sheath of claim 4 wherein said locking mechanism comprises:
a rack that is displaceable;
a pinion that engages and drives said rack, said pinion being manipulated by an operator; and
a compressible gland surrounding said first catheter, said gland flexing inward against said first catheter to prevent its movement whenever said rack is driven against said gland.

6. The steerable sheath of claim 1 wherein said first distal end is atraumatic.

7. The steerable sheath of claim 1 wherein said first distal end is radiopaque.

8. The steerable sheath of claim 6 wherein said second distal end is atraumatic.

9. The steerable sheath of claim 6 wherein said second distal end is radiopaque.

10. The steerable sheath of claim 1 wherein said composite material comprises a polymer and an additive.

11. The steerable sheath of claim 10 wherein the polymer is nylon.

12. The steerable sheath of claim 10 wherein the polymer is polyester.

13. The steerable sheath of claim 10 wherein said composite comprises nylon 12, 25% talc and 10% barium sulfate.

14. The steerable sheath of claim 1 wherein said first material comprises a polymer that is reinforced with stainless steel braid.

15. The steerable sheath of claim 1 wherein said first catheter comprises at least one aperture along its length, said aperture providing fluid communication with said lumen of said second catheter.

16. The steerable sheath of claim 4 wherein said first catheter includes curve indicia thereon, said curve indicia permitting the operator to manipulate said first catheter with respect to said locking mechanism to form a desired curve at said first distal end.

17. A steerable sheath comprising:
a first catheter including a first lumen and a second catheter including a second lumen, said second catheter being slidable and rotatable within said first lumen; and
a locking mechanism coupled to said first catheter and to said second catheter, said locking mechanism preventing or permitting said first catheter to slide or rotate within said second catheter.

18. The catheter of claim 17 wherein said first catheter comprises a first distal end having a first radius of curvature and comprising a first material, said second catheter comprises a composite material and further comprises a second distal end, and wherein said composite material is more rigid than said second material, said second distal end being able to form a plurality of curve shapes when said first lumen is slid or rotated within said second lumen and wherein said first distal end projects from said second distal end.

19. The steerable sheath of claim 18 wherein a portion of said second catheter adjacent said second distal end is linear or has a second radius of curvature, said second radius of curvature being different from said first radius of curvature.

20. The steerable sheath of claim 18 wherein a portion of said second catheter adjacent said second distal end is linear or has a second radius of curvature, said second radius of curvature being similar to said first radius of curvature.

21. The steerable sheath of claim 18 wherein said first distal end is atraumatic.

22. The steerable sheath of claim 18 wherein said first distal end is radiopaque.

23. The steerable sheath of claim 21 wherein said second distal end is atraumatic.

24. The steerable sheath of claim 21 wherein said second distal end is radiopaque.

25. The steerable sheath of claim 18 wherein said composite material comprises a polymer and an additive.

26. The steerable sheath of claim 25 wherein the polymer is nylon.

27. The steerable sheath of claim 25 wherein the polymer is polyester.

28. The steerable sheath of claim 25 wherein said composite comprises nylon 12, 25% talc and 10% barium sulfate.

29. The steerable sheath of claim 18 wherein said first material comprises a polymer that is reinforced with stainless steel braid.

30. The steerable sheath of claim 17 wherein said locking mechanism comprises:
a rack that is displaceable;
a pinion that engages and drives said rack, said pinion being manipulated by an operator; and
a compressible gland surrounding said first catheter, said gland flexing inward against said first catheter to prevent its movement whenever said rack is driven against said gland.

31. The steerable sheath of claim 18 wherein said first catheter comprises at least one aperture along its length, said aperture providing fluid communication with said lumen of said second catheter.

32. The steerable sheath of claim 18 wherein said first catheter includes curve indicia thereon, said curve indicia permitting the operator to manipulate said first catheter with respect to said locking mechanism to form a desired curve at said first distal end.
